(19) United States
(12) Patent Application Publication
(10) Pub. No.: US 2006/0173413 A1
(43) Pub. Date: Aug. 3, 2006

(54) Intravenous catheter insertion device with retractable needle

(75) Inventor: Tzu-Sheng Fan, Miao Li Hsien (TW)

Correspondence Address: BACON & THOMAS, PLLC, 625 SLATERS LANE, FOURTH FLOOR, ALEXANDRIA, VA, 22314, US

(73) Assignee: TAIJECT MEDICAL DEVICE CO., LTD., MIAOLI (TW)

(21) Appl. No.: 11/016,826

(22) Filed: Dec. 21, 2004

(51) Int. Cl. A61M 5/20; A61M 5/178

(52) U.S. Cl. 604/157; 604/164.04

(57) ABSTRACT

An intravenous catheter insertion device is disclosed to include a barrel, which has a body and a stop wall, a deformable portion extending from the body, a retainer connected with the deformable portion, and a press portion for pressing by the user to deform the deformable portion. A needle set, which has a cylindrical needle holder, a needle fastened to the needle holder at the front side, a springy stop portion extending from the needle holder, and a retainer connected with the needle holder near the front end of the needle holder. And a spring member, which is mounted around the needle set inside the barrel, and has a first end stopped against the stop wall of the barrel and a rear end stopped against the springy stop portion of the needle set.

### BACKGROUND OF THE INVENTION

1. Field of the Invention

The present invention relates generally to medical implement and more specifically, to an intravenous catheter insertion device with retractable needle.

2. Description of the Related Art

Various intravenous catheter insertion devices are well known. These devices commonly have a needle set made retractable to prevent contamination accidentally. One conventional design of intravenous catheter insertion device is known having a spring plate provided at the needle holder and a retaining hole provided at the barrel for engagement with the spring plate. There is known another prior art design, which has the lock control provided at the barrel and the retaining hole provided at the needle set. This design is complicated, not convenient in use. Further, the user cannot visually check if the intravenous catheter has been accurately inserted into the patient's vein or not.

### SUMMARY OF THE INVENTION

The present invention has been accomplished under the circumstances in view. It is the main object of the present invention to provide an intravenous catheter insertion device with retractable needle, which has a simple, steady structure for convenient use. It is another object of the present invention to provide an intravenous catheter insertion device with retractable needle, which allows the user to visually check accurate installation of the intravenous catheter.

To achieve these and other objects of the present invention, the intravenous catheter insertion device comprises a barrel, the barrel having a body, a chamber defined within the body, a stop wall at a front end of the body in front of the chamber, a deformable portion connected with the body, a retainer connected with the deformable portion, and a press portion for pressing by the user to deform the deformable portion; a needle set axially movable in and out of the barrel, the needle set having a cylindrical needle holder, a receiving hole axially extending through the cylindrical needle holder, a needle fastened to the needle holder and axially forwardly extending out of a front end of the needle holder, a springy stop portion extending from the needle holder, and a retainer connected with the needle holder near the front end of the needle holder; and a spring member mounted around the needle set inside the chamber of the barrel, the spring member having a first end stopped against the stop wall of the barrel and a second end stopped against the springy stop portion of the needle set.

### BRIEF DESCRIPTION OF THE DRAWINGS

FIG. 1 is an exploded view of an intravenous catheter insertion device according to the present invention.

FIG. 2 is a front view in section of FIG. 1.

FIG. 3 is a front view of FIG. 1.

FIG. 4 is a top view of a part of the present invention.

FIG. 5 is a sectional assembly view of the intravenous catheter insertion device according to the present invention.

FIG. 6 is a schematic drawing showing the use of the present invention.

FIG. 7 is a sectional view showing the needle set received inside the barrel after disconnection from the intravenous catheter.

### DETAILED DESCRIPTION OF THE INVENTION

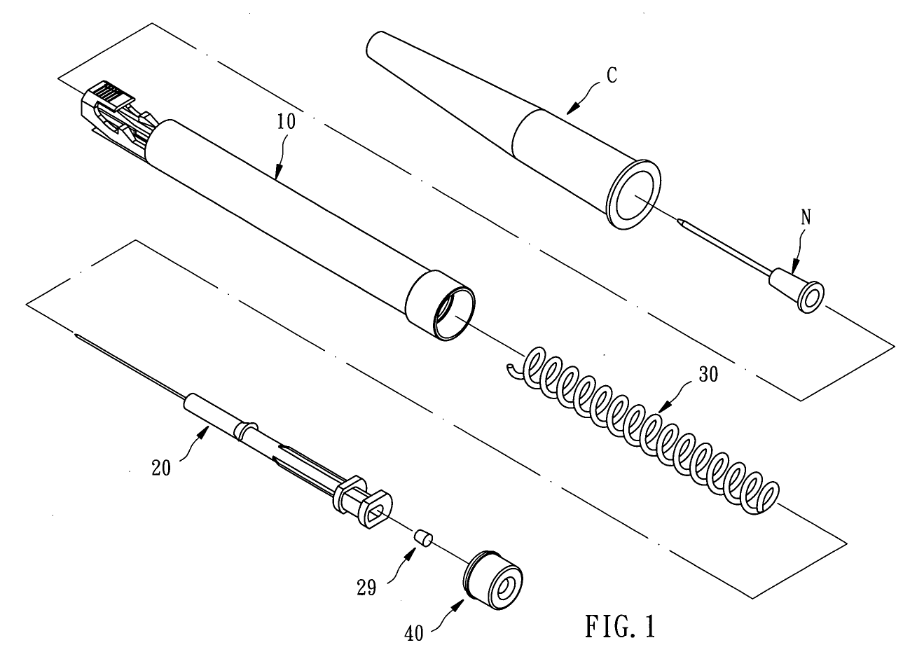

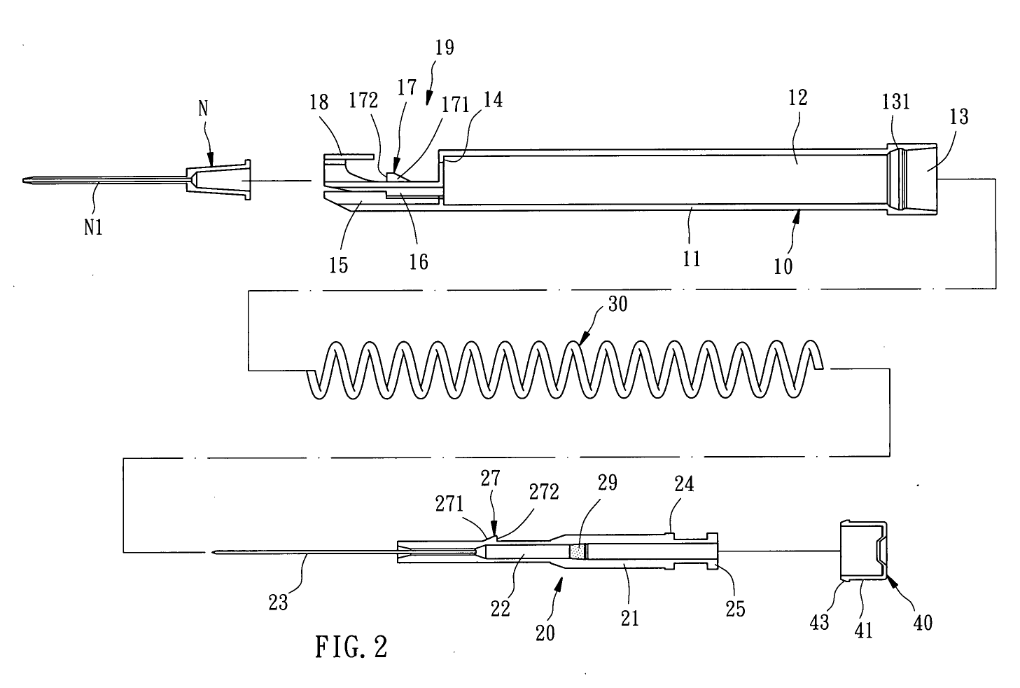

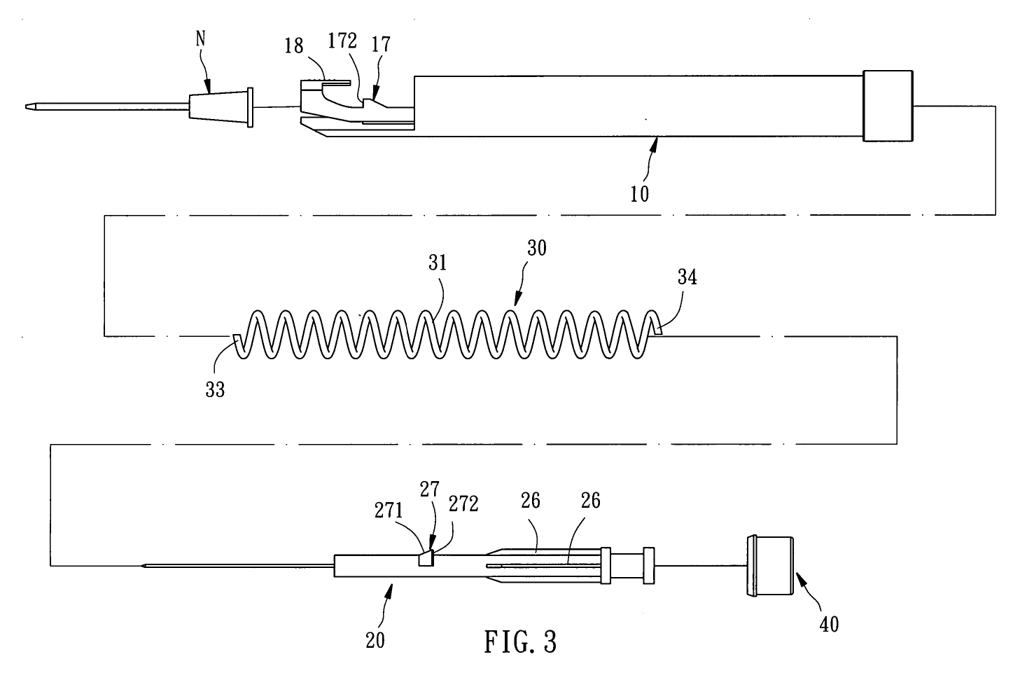

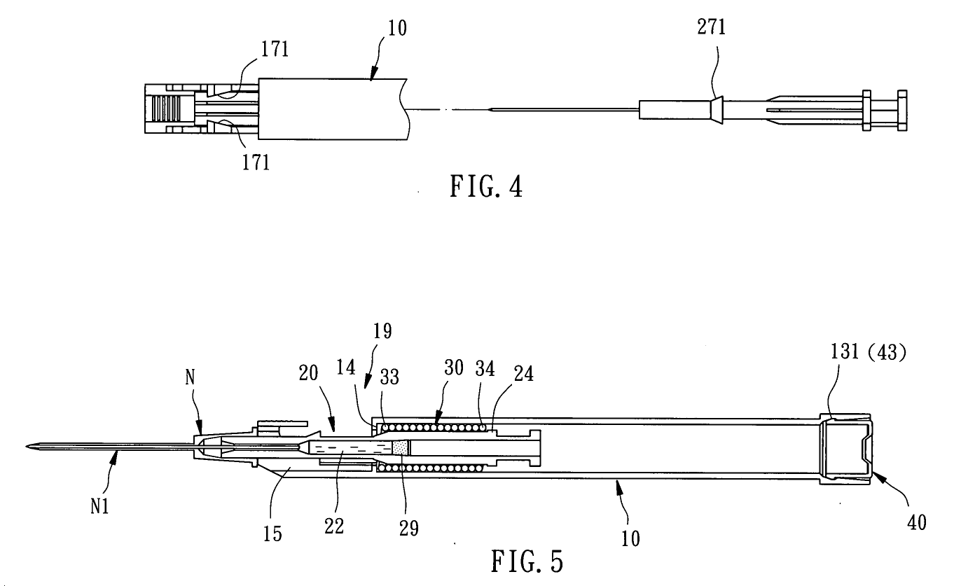

Referring to FIGS. 1˜5, an intravenous catheter insertion device is adapted to install an intravenous catheter N having a flexible needle cannula N1 in the vein of a patient. The intravenous catheter insertion device comprises:

A barrel 10 has a body 11, a chamber 12 defined within the body 11, a mounting hole 13 at one end, namely, the rear end of the body 11 in communication with the chamber 12, a retaining portion 131 disposed inside the mounting hole 13, a stop wall 14 at the other end, namely, the front end of the body 11, a support 15 axially forwardly extending from the front end of the body 11, two deformable portions 16, two retainers 17 respectively formed integral with the deformable portions 16, each retainer 17 has a sliding face 171 and a stop face 172, a press portion 18, and an opening 19 for enabling the user to visually check the area around the retainers 17.

A needle set 20 has a cylindrical needle holder 21, a receiving hole 22 axially extending through the needle holder 21, a needle 23 fastened to the needle holder 21 and axially forwardly extending out of the front end of the needle holder 21, a springy stop portion 24 extending around the periphery of the needle holder 21, an end flange 25 at the rear end of the needle holder 21, a plurality ribs 26 axially and equiangularly spaced around the periphery of the needle holder 21, a retainer 27 formed integral with the periphery of the needle holder 21 near the front end, and an insert 29 inserted into the receiving hole 22. The retainer 27 is shaped like a half cone, having a sliding face 271 and a stop face 272.

A spring member 30 is a compressible spiral spring and having a spring body 31 mounted around the needle holder 20, a first stop portion 33 disposed at one end of the spring body 31 and stopped against the stop wall 14 of the barrel 10, and a second stop portion 34 stopped against the springy stop portion 24 of the needle set 20.

A seal 40 has a body 41 inserted into the mounting hole 13 of the barrel 10, an annular stop flange 43 extended around the periphery of one end of the cap body 41 and engaged with the retaining portion 131 of the barrel 10.

Referring to FIG. 5, the spring member 30 is inserted into the inside of the chamber 12 to stop the first stop portion 33 against the stop wall 14 of the barrel 10, and then the needle set 20 is inserted through the spring member 30 with the springy stop portion 24 stopped against the second stop portion 34 of the spring member 30, and then the needle set 20 is pushed forwards to compress the spring member 30 and to move the sliding face 271 over the sliding face 171, for enabling the stop face 272 to be stopped against the stop face 172. Therefore, the intravenous catheter N is attached to the support 15, and then the protective cap C is capped on the barrel 10 to protect the intravenous catheter N and the needle set 20.

The use of the present invention is explained hereinafter. After removal of the protective cap C from the barrel 10, the flexible needle cannula N1 is inserted with the needle 23 of the needle set 20 into the patient's vein. Through the opening 19, the user can visually check flowing of blood from the patient's body into the needle 23 and the flexible needle cannula N1, assuring accurate installation of the intravenous catheter N.

Thereafter, apply a pressure to the patient's body with one hand to hold down the flexible needle cannula N1 in the patient's vein, and then pull the barrel 10 with the other hand backwards to separate the needle set 20 from the intravenous catheter N and the patient's vein.

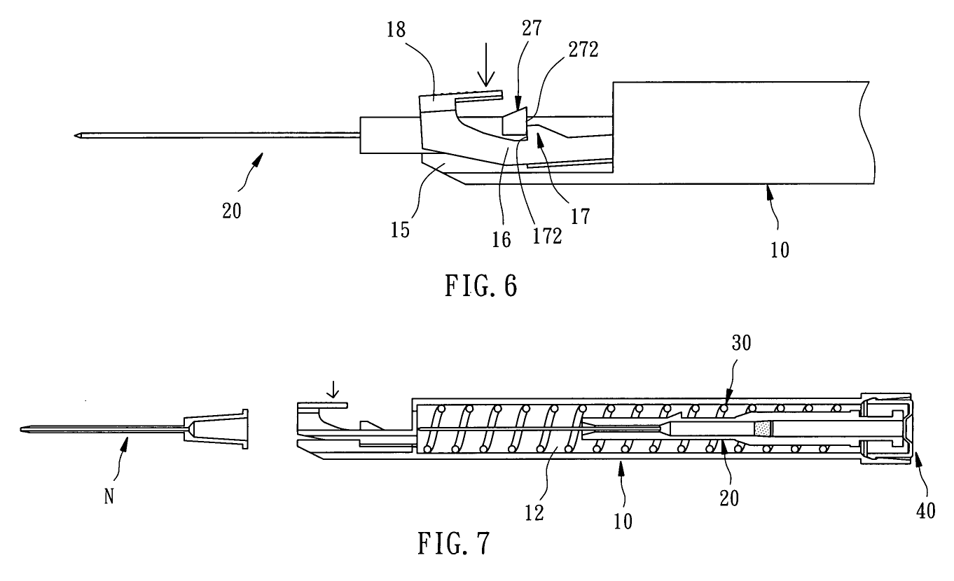

As shown in FIG. 6, after separation of the device from the intravenous catheter N, press the press portion 18 to deform the deformable portions 16 and to further disengage the two retainers 17 from the retainer 27 of the needle set 20. Upon disconnection of the retainers 17 of the barrel 10 from the retainer 27 of the needle set 20, the spring member 30 immediately returns to its former shape and to pull the needle set 20 backwards to the inside of the chamber 12 inside the barrel 10.

After the needle set 20 has been received inside the chamber 12, the seal 40 is fastened to the mounting hole 13 of the barrel 10 to prevent insertion of an external body into the barrel 10 to push the needle set 20 out of the barrel 10 (see FIG. 7).

In the aforesaid preferred embodiment of the present invention, the retainer of the needle set is shaped like a half cone. However, this shape is not a limitation, i.e. the retainer of the needle set can be made having any of a variety of shapes.

As indicated above, the invention provides an intravenous catheter insertion device, which has the following features:

1. The whole structure of the intravenous catheter insertion device is simple and steady for convenient use.

2. Through the opening at the support of the barrel, the user can visually check accurate installation of the intravenous catheter.

What is claimed is:

1. An intravenous catheter insertion device comprising:
a barrel, said barrel having a body, a chamber defined within said body, a stop wall at a front end of said body in front of said chamber, a deformable portion connected with said body, a retainer connected with said deformable portion, and a press portion for pressing by the user to deform said deformable portion;
a needle set axially movable in and out of said barrel, said needle set having a cylindrical needle holder, a receiving hole axially extending through said cylindrical needle holder, a needle fastened to said needle holder and axially forwardly extending out of a front end of said needle holder, a springy stop portion extending from said needle holder, and a retainer connected with said needle holder near the front end of said needle holder; and
a spring member mounted around said needle set inside said chamber of said barrel, said spring member having a first end stopped against said stop wall of said barrel and a second end stopped against said springy stop portion of said needle set.

2. The intravenous catheter insertion device as claimed in claim 1, wherein said needle set further has an end flange extending around a rear end of said needle holder.

3. The intravenous catheter insertion device as claimed in claim 1, wherein said needle set has a plurality of ribs axially spaced around the periphery of said needle holder.

4. The intravenous catheter insertion device as claimed in claim 1, wherein said retainer of said needle set has a conical shape.

5. The intravenous catheter insertion device as claimed in claim 1, wherein said spring member is a compressible spiral spring.

6. The intravenous catheter insertion device as claimed in claim 1, wherein said barrel has a mounting hole in a rear end of said body thereof in communication with said chamber.

7. The intravenous catheter insertion device as claimed in claim 6, further comprising a seal adapted to seal said chamber of said barrel, said seal having a body insertable into said mounting hole of said barrel.

8. The intravenous catheter insertion device as claimed in claim 7, wherein said barrel has a retaining portion for securing said seal.

9. The intravenous catheter insertion device as claimed in claim 8, wherein said seal has an annular stop flange extending around the periphery of said body thereof for engaging said retaining portion of said barrel.

10. The intravenous catheter insertion device as claimed in claim 1, wherein said barrel has an opening around said deformation portion and said press portion for enabling the user to visually check flowing of a fluid in said needle set.
